(19) United States
(12) Patent Application Publication
(10) Pub. No.: US 2014/0025167 A1
(43) Pub. Date: Jan. 23, 2014

(54) SYSTEM AND METHOD OF CERVICAL DISTRACTION FOR REOPERATIVE PROCEDURES

(71) Applicant: Nicholas Poulos, Belleville, IL (US)

(72) Inventor: Nicholas Poulos, Belleville, IL (US)

(21) Appl. No.: 13/553,346

(22) Filed: Jul. 19, 2012

(51) Int. Cl. A61F 2/44

(52) U.S. Cl. 623/17.16

(57) ABSTRACT

A system is disclosed for distracting a disc space in a cervical spine at a level adjacent to a previously operated disc space where, in the previous surgery, a cervical plate has been applied to the anterior of the spine to span the previously operated upon disc space and where the cervical plate is attached to the vertebrae bodies on opposite sides of the previously operated upon disc space with the cervical plate covering at least a portion of the vertebrae bodies. A conventional distractor post is screwed into a third vertebrae body, and a second distractor post or member is fixedly and removably secured to the cervical plate. A conventional distractor instrument is applied to the posts of both distractor posts for applying a distraction force to the vertebrae bodies delimiting the disc space to be operated upon. A surgical method of distraction is also disclosed.

### CROSS-REFERENCE TO RELATED APPLICATIONS

Not applicable.

### STATEMENT REGARDING FEDERALLY SPONSORED RESEARCH OR DEVELOPMENT

Not Applicable.

### BACKGROUND OF THE DISCLOSURE

This disclosure relates to a common and challenging problem faced by spine surgeons is reoperation on a previously operated cervical spine for adjacent segment disease. Typically, the patient will have undergone an anterior cervical discectomy, interbody fusion, and plate stabilization at the index procedure. Many years later, such patients may develop compressive pathology at the adjacent level that is symptomatic and unresponsive to medical treatment including epidural injections. The etiology of this is probably multifactorial, but likely includes disease progression as well as induced hypermobility from the adjacent fusion causing accelerated breakdown of the adjacent disc space. Such reoperation for adjacent segment disease has often required removing the old cervical plate, decompressing the new level, and then plating the new level. The reason for plate removal from the index surgery is that it frequently abutted or prevented placement of a new plate, that is, the plates would overlap and not sit flush to the spine permitting screw fixation.

The above scenario had many deleterious ramifications. The surgeon would have to dissect not only at the new level of pathology but the previously operated surgical corridor as well. Normally, the approach involves an interfascial dissection technique dividing the superficial, middle, and deep cervical fascia sequentially to reach the prevertebral space staying medial to the carotid artery and lateral to the trachea and the esophagus. The anterior cervical spine is easily and quickly identified. The majority of this is accomplished by blunt finger dissection as the approach utilizes natural anatomic dissection planes. This advantage is completely lost on a reoperation as all the dissection planes have scarred down. Now the surgeon must use meticulous sharp and blunt dissection technique with care taken by the surgeon to make sure the critical structures of the neck are not damaged (e.g., the carotid artery, trachea, and esophagus).

Trying to limit the amount of dissection in such a challenging and unforgiving environment, zero-profile plate cervical interbody implants were designed. There are many manufactures and many styles of such zero-profile plating systems, but they share a few common features. The structural implant and plate are one unit with the plate attached to the implant anteriorly. Additionally the plate is exactly the height of the implant, or put another way, the desired disc space height, which is established on reconstruction. The implant has a central hollow core, which allows for bone grafting and fusion. Finally, screws are placed through the plate and implant into the cervical vertebral bodies for fixation and stabilization. With such zero-profile plating systems, the zero-profile screws converge to the midline thus avoiding screws from the cervical plate which will be in a more lateral position. The end result of this design is that no portion of the plate extends superiorly or inferiorly from the newly fused disc space exists along the ventral surface of the spine to contact, abut, or impinge on the previously placed plate. Therefore, the surgeon can generally limit dissection to the new surgical level knowing that he will not have to remove an old plate. This helps minimize the chances of injury to the previously mentioned critical structures, although the surgeon usually has to contend with some scar tissue. One example of such a zero-profile plate cervical interbody implant is the AVS® Anchor-C Cervical Cage System commercially available from Stryker Spine of Kalamazoo, Mich.

Although such zero-profile plate systems solve problems of fixation in reoperative cervical spine surgery, there still exists a critical step in the dissection process that can pose a significant barrier to utilizing this technology. Specifically, before the zero-profile implant can be applied, a standard discectomy and decompression of the spinal cord and nerve roots must be performed. This requires that the diseased disc to undergo reoperative surgery be distracted. Conventionally, distractor posts are impacted and screwed into the vertebral bodies above and below the disc space of interest such that the distraction posts are generally perpendicular to the spine in the midline. A cervical distractor is then applied to the posts, and the disc space is distracted developing a surgical corridor for discectomy and for endplate preparation for fusion, as well as optimizing visualization of the spinal cord and nerve roots for decompression. This maneuver also allows height to be restored to the disc space if this is part of the surgical strategy . . . . This is shown in FIGS. 2-6 of the present disclosure.

However on reoperation at an adjacent symptomatic level, the ability to place posts to distract the new disc space is significantly compromised because, in many instances, the previously applied cervical plate blocks satisfactory placement of a distraction post in the vertebral body that has been plated. Placing a distraction post in the midline immediately adjacent to plate may not be possible because of lack of space, or if possible, post trajectory into vertebral body invariably intersects a significant portion of future zero-profile screw trajectory thus weakening the bone. As a result, fixation for the zero-profile screw is suboptimal, if even achievable. Alternatively, placement of the distraction post lateral to the plate is associated with multiple problems. Placement of the post perpendicular to the spine risks breaching the bone and injuring important structures, for example, nerves or the vertebral artery. Altering the post trajectory from perpendicular to lateral to medial will fixate the spine but at the cost of having the free end of the post protruding into the soft tissue bank that has been retracted medially potentially injuring the trachea or esophagus. Additionally, with-medially extending posts, application of the cervical distractor is problematic as it is very difficult to access the free end of the post which is likely buried in soft tissue and even if this could be finessed, the second post in the vertebral body on the other side of the disc space will not be parallel which prohibits use of the standard cervical distractor whose functionality requires the two posts to be generally parallel.

### SUMMARY OF THE DISCLOSURE

The present disclosure discloses a novel distractor post with applicator, which represents an improvement to existing cervical distractors by facilitating the use of any zero-profile plate cervical interbody implant with any previously applied anterior cervical plate in the context of reoperative cervical spine surgery. Additionally, a minor modification to existing anterior cervical plates is also described that enhances the use and effectiveness of the novel distractor post.

In accordance with the present disclosure, a system is disclosed for distracting a disc space in a cervical spine at a level adjacent to a previously operated disc space where, in the previous surgery, a cervical plate has been applied to the anterior spine to span the previously operated upon disc space and where the cervical plate is attached to the vertebrae bodies on opposite sides of the previously operated upon disc space with the cervical plate covering at least a portion of the vertebrae bodies. A threaded distractor post is threadably secured to a third vertebrae body delimiting the disc space to be operated upon. A distractor member is fixedly and removably secured to the cervical plate. Both the distractor post and the distractor member have a post extending generally perpendicularly to the midline of the cervical spine and projecting anteriorly of the cervical spine so that a conventional distractor instrument may be applied to the posts of the distractor post and the distractor member for applying a distraction force to the vertebrae bodies delimiting the disc space of interest.

A method is disclosed for distracting a disc space in the cervical spine at an adjacent symptomatic level to a disc space that has been previously operated upon where in such previous operation a cervical plate was secured to the anterior of the cervical vertebrae bodies on opposite sides of the previously operated upon disc space. The cervical plate covers at least a portion of the anterior vertebral body of the vertebrae delimiting the disc space to be operated upon. The method of the present disclosure comprises: installing a threaded distraction post in another vertebrae body delimiting the disc space to be operated upon; removably attaching a distractor member to the cervical plate, where both the distractor post and the distractor member have a post extending anteriorly from approximately the midline of the cervical spine; applying a distractor instrument to the posts; and operating the operating the distractor instrument so as to distract the disc space to be operated upon.

Among the many features and advantages of the system and method of the present disclosure may be noted the provision of a system that may be used with conventional cervical distractor systems or instruments so that such system and method are of nominal cost to implement;

The provision of such a system and method in which the novel distractor member is disposable;

The provision of such a novel distractor member that may be readily removably attached to a previously installed cervical plate installed of the vertebrae body above or below the adjacent segment undergoing reoperative surgery without the necessity of having to remove such previously installed plate;

The provision of such a novel distractor member and method where the distractor member may be removably attached to the previously installed cervical plate in such manner that such attachment does not cause the screws fixing the cervical plate to the vertebrae bodies to pull out of the vertebrae body;

The provision of such a novel distractor member and method that may be used in conjunction with a conventional distraction post installed on the adjacent vertebrae body that does not have a cervical plate attached thereto;

The provision of such a novel distractor member and method that may be utilized in situations where, if the disc space to be distracted is between cervical plates applied to the vertebrae bodies above and below the disc space undergoing reoperative surgery, two of the distractor members of the present disclosure may be removably attached to the two adjacent cervical plates above and below the disc space undergoing reoperative surgery thus enabling distraction of the disc space without removal of or damage to the cervical plates;

The provision of such a novel distractor member and method that facilitates distraction of an adjacent segment where, rather than a conventional distraction post being imbedded in the vertebrae body proximate to a previously fused disc space, the distraction member of the present disclosure applies distraction force to the vertebrae bodies via the bone screws holding the cervical plate to its respective vertebrae body;

The provision of such a novel system and method that is easy to use, does not require special training, and does not require the removal and reinstallation of cervical plates applied in previous cervical spine surgeries.

Other objects and features of the system and method of the present disclosure will be in part apparent and in part pointed out hereinafter.

### BRIEF DESCRIPTION OF THE DRAWINGS

FIG. 1 is an anterior lateral view of a cervical spine with symptomatic cervical disc disease at C4/C5;

FIG. 2 is an anterior view of a cervical spine undergoing discectomy, decompression, and fusion to treat a diseased disc, for example between C4/C5, where conventional distraction posts have been placed in the vertebrae bodies delimiting the C4/5 disc space and where the vertebrae have been rotated slightly so as to better show insertion of the posts into the vertebrae;

FIG. 3 is a lateral view of FIG. 2 illustrating the installation of the conventional distraction posts into the anterior of the cervical spine;

FIG. 4 illustrates a cervical distractor instrument applied to posts shown in FIG. 3 for distracting the disc space between C4/C5 so as to increase disc space height to facilitate discectomy, endplate preparation for fusion, and decompression of spinal cord and nerve roots;

FIG. 5 is a view similar to FIG. 4 wherein the spinal cord and nerve roots have been decompressed, and the disc space has been reconstructed with an interbody implant installed in the evacuated disc space with the central hollow core of the implant filled with bone graft for fusion of the adjacent vertebrae bodies;

FIG. 6 is an enlarged, detailed view taken along line 6-6 of FIG. 5;

FIG. 7 is an anterior view of the cervical vertebrae bodies shown in FIGS. 2-6 after decompression and interbody fusion where an anterior cervical plate has been affixed to the vertebrae bodies delimiting the diseased disc space for internal fixation and stabilization, and thus enhancing fusion;

FIG. 8 is an enlarged view taken along line 8-8 of FIG. 7 illustrating the cervical plate;

FIG. 9 is a view similar to FIG. 3 illustrating a previously operated cervical spine where, for example, the disc between C4/C5 has previously undergone anterior cervical discectomy, interbody fusion, and plate stabilization, where an adjacent segment symptomatic cervical disc disease has developed at an adjacent level, for example between C5/C6, where FIG. 9 illustrates the installation of a standard distractor post affixed to C6 in the conventional manner and illustrates a distraction member in accordance with the present disclosure removably secured or applied to an anterior cervical plate spanning an adjacent previously surgically treated disc space, for example C4/C5, where the installation of the distraction member does not require the removal of the cervical plate, such that a distractor instrument may be applied to the posts of both the distractor post and the distractor member so as to distract the disc space undergoing surgery;

FIG. 10 is a lateral view of FIG. 9 illustrating features of the distraction member of the present disclosure having two spaced blades or tangs on its distal end with a space therebetween for receiving an edge of the cervical plate, where the lower blade has a chisel edge that may be readily impacted (driven) underneath a proximate edge of the cervical plate so as to rigidly capture the plate between the blades thus solidly, but removably attaching the distraction member to the cervical plate thus permitting distraction of the disc space without necessitating the removal of the cervical plate:

FIG. 11 illustrates the use of a conventional cervical distractor instrument applied to the distractor posts on opposite sides of the disc space to be distracted so that distraction forces may be applied to the posts of the conventional distractor post and the distractor member of the present disclosure removably attached to the cervical plate anchored to the adjacent vertebrae by its four screws;

FIG. 12 is an anterior view of a cervical spine similar to FIG. 11 having a commercially available cervical interbody implant applied to the previously operated adjacent segment, and having a zero-profile plate system applied to the disc space undergoing surgery;

FIG. 13 is a lateral view of the cervical spine shown in FIG. 12;

FIGS. 14a-14d are various views of the distractor member of the present disclosure;

FIGS. 15a-15e illustrate a post applicator of the present disclosure for installing the distractor member of the present disclosure on a cervical plate, as illustrated in FIGS. 9 and 10;

FIGS. 16a-16e illustrate, on an enlarged scale, different views of the working end of the post applicator and driver shown in FIGS. 15a-15i;

FIG. 17 is a view of the applicator/driver of the present disclosure shown in FIGS. 15a-15e having a distractor member or post of the present disclosure inserted in the operative end of the applicator for installation of the member onto a previously applied cervical plate, where the applicator and the member are inclined relative to the plate so that the lower blade of the distractor member may be inserted under the edge of the plate;

FIG. 18 is a view similar to FIG. 17 with the applicator rotated so that the upper and lower blades of the distractor member are positioned to receive the edge of the plate such that upon the surgeon hitting the pommel end of the applicator, the lower blade will be driven under the plate and the edge of the plate will be received between the blades so that the plate is captured between the blades;

FIG. 19 is an enlarged view of the end of the applicator, the post, and the edge of the plate, as they are shown in FIG. 18;

FIGS. 20a-20f are various views of a modified anterior cervical plate of the present disclosure having a groove in the underside of each minor end so that a distraction member of the present disclosure may be readily installed on the cervical plate should symptomatic adjacent segment disease develop in the future;

FIG. 21 is a view similar to FIG. 18 illustrating the application of a distraction member of the present disclosure onto an edge of the cervical plate shown in FIG. 20 where a portion of a lower blade is at least partly received within the slot in the underside of the plate, as shown in FIG. 20; and

FIG. 22 is an enlarged view of the end of the applicator, the post, and the edge of the plate, as they are shown in FIG. 21.

Corresponding reference characters indicate corresponding components throughout the several views of the drawings.

### DESCRIPTION OF PREFERRED EMBODIMENTS

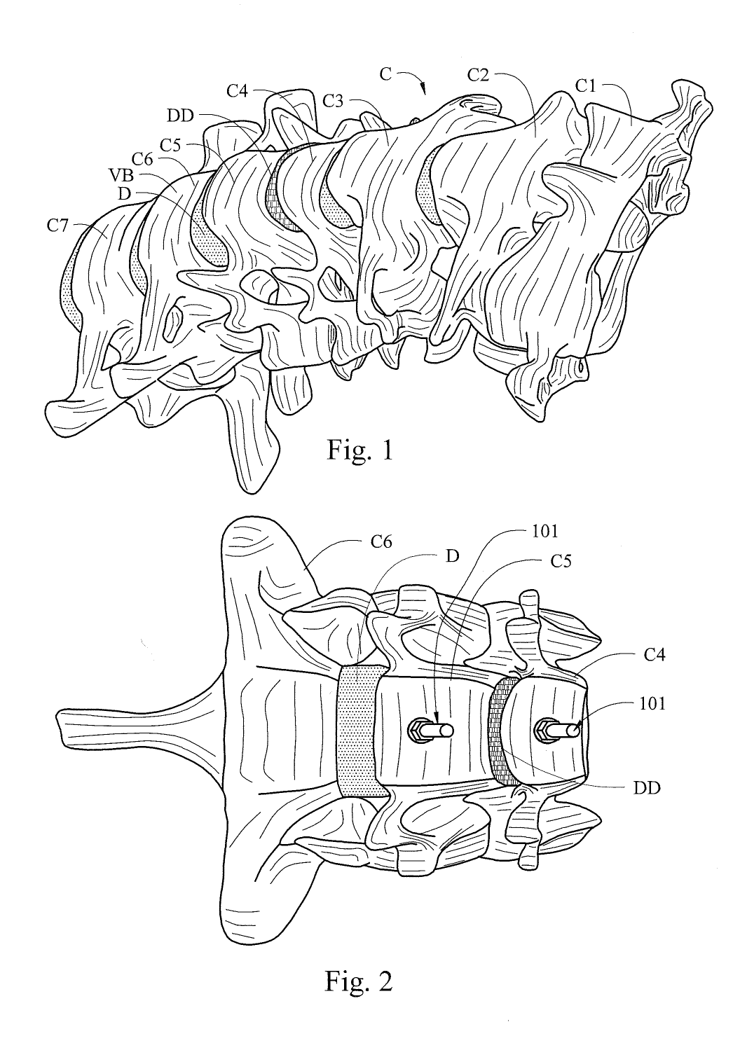

Referring now to the drawings, and particularly to FIG. 1, a perspective view of the cervical portion of the spine is indicated in its entirety at C having seven vertebrae bodies VB indicated by conventional nomenclature C1-C7. A disc, as generally indicated at D, is located between each of the adjacent cervical vertebrae bodies.

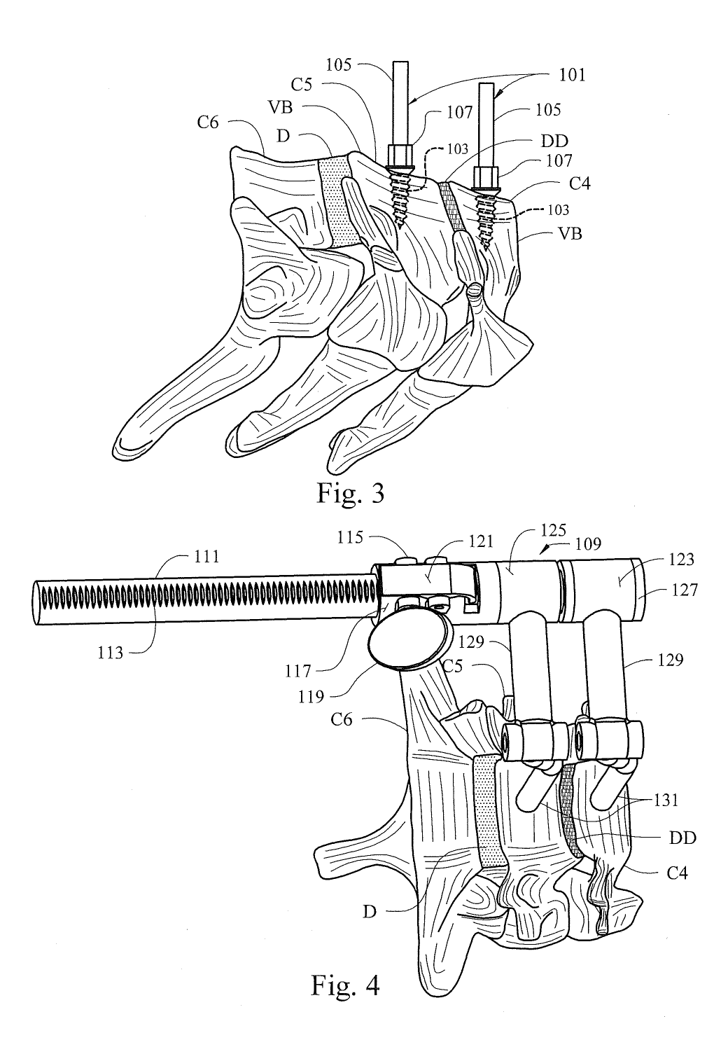

As shown in FIGS. 2 and 3, if a disc D becomes diseased and symptomatic, as for example between C4 and C5, in accordance with current surgical practice, the vertebrae bodies VB above and below the diseased disc (i.e., the vertebrae bodies delimiting the diseased disc) may be fused. In such spinal fusion surgery, it is conventional to thread conventional distractor posts 101 into the vertebrae bodies on opposite sides of the disc undergoing surgery along the midline of the vertebrae bodies. Each of these conventional distractor posts has a threaded screw end 103 that may be threaded into the bony structure of its respective vertebrae body and a post 105 extending axially from the screw end such that the post extends generally perpendicular to the midline, as shown in FIG. 3. Such distractor posts typically have a hexagonal body 107 proximate the transition between the screw end and the axial post such that an appropriate tool (e.g., a socket) may be used to screw the distractor post into the vertebrae body.

Then, as shown in FIGS. 4-6, a conventional rack and pinion distractor instrument 109 may be applied to the spaced distractor posts 105 to distract the disc space a desired amount. Distractor instrument 109 typically has an elongate bar 111 having a rack of teeth 113 extending along the bar. A pinion 115 having pinion teeth (not shown) that engage rack teeth 113 is rotatably supported in a housing 117 so that upon rotation of the pinion via a thumb screw 119 rotated by the surgeon, the rack teeth and the pinion teeth cooperate so as to effect axial movement of the housing 117 along the length of bar 111. The width of the thumb screw 119 relative to the diameter of pinion 115 gives a significant mechanical advantage sufficient to distract the disc space. A pawl 121 is pivotally mounted on housing 117 and is spring biased into engagement with the rack teeth 113 so as to lock the housing in a fixed position thereby to maintain a desired amount of distraction. Of course, by depressing the end of the pawl adjacent the pinion, the pawl is lifted clear of the rack teeth thus unlocking pinion from the rack.

The distractor 109 further has pair of distractor bodies, as indicated at 123, 125, mounted on bar 111. Distractor body 123 is shown to be mounted on the distal end of the bar 111 and it abuts a flange 127 affixed to the end of the bar thus preventing movement of the body 123 past the flange. Distractor body 125 is operatively coupled to pinion body 117 such that upon rotating thumb screw 119, the housing 117 and distractor body 125 are moved together in axial direction along bar 111. In this manner, distractor body 125 may be forcefully moved in axial direction along bar 111 toward and away from distractor body 123. As indicated at 129, each of the distractor bodies has an arm extending perpendicularly to the distractor body and a distractor post tube 131 is pivotally carried on the outer end of the arm. Each of the distractor post tubes is adapted to receive a respective post 105 of the distractor posts 101 imbedded in the adjacent vertebrae bodies VB, as shown in FIG. 3. With the posts 105 received in tubes 131, the surgeon may operate the pinion 115 to move the housing 125 away from housing 123 thus applying a force on the adjacent vertebrae bodies to as to distract the disc space therebetween. When the disc space has been distracted a desired amount, the spring biased pawl 121 will lock the distractor and will thus maintain the desired amount of distraction.

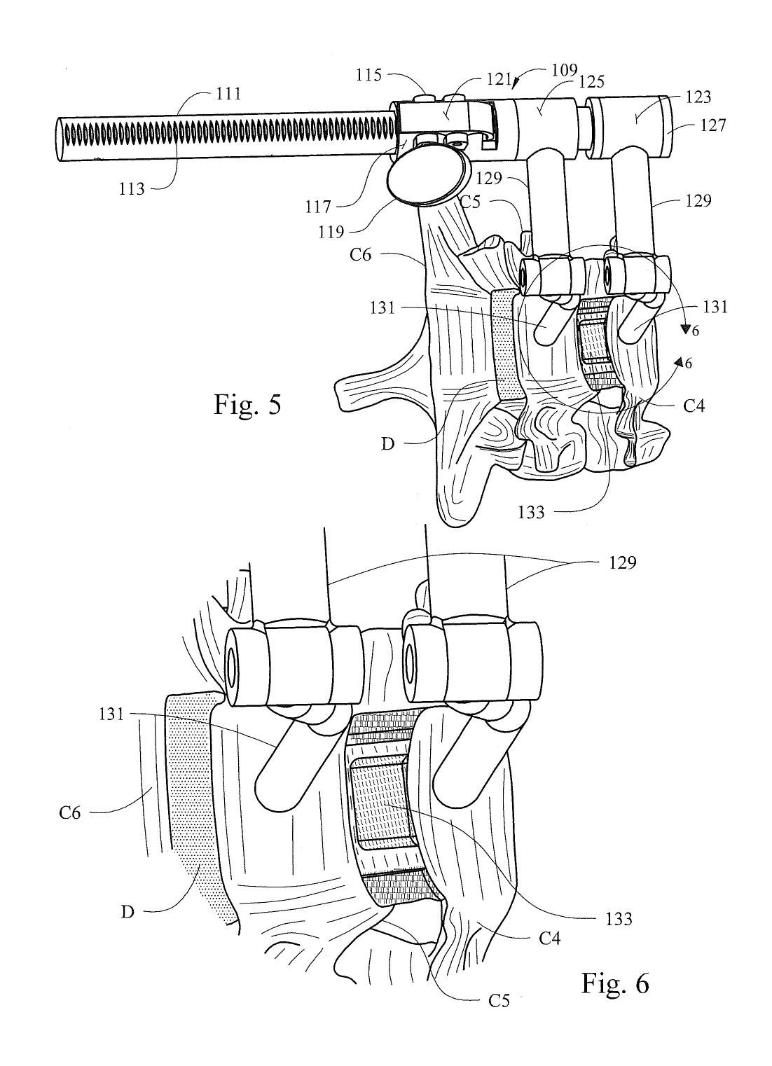

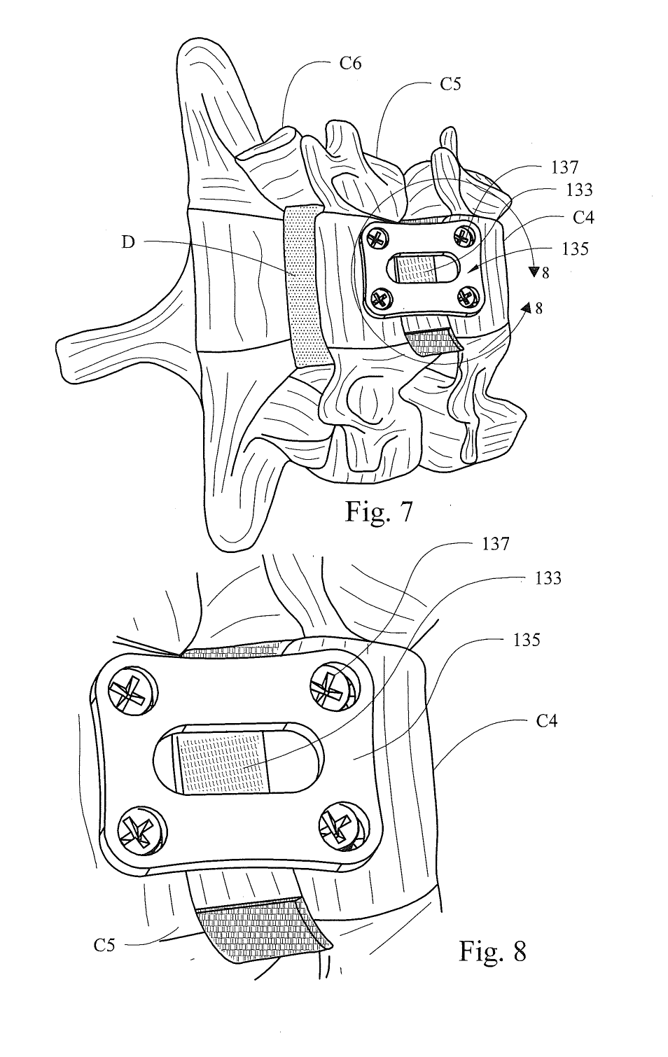

As shown in FIGS. 5 and 6, after distraction of the disc space, discectomy may be performed to evacuate the disc space, decompression of the spinal cord and nerve roots is carried out, and the endplates of the vertebrae bodies may be prepared in the conventional manner. A conventional implant 133 may be installed in the evacuated disc space so as to maintain the desired disc space. Bone graft material may be packed into the central hollow core of the implant so as to promote fusion of the adjacent vertebrae bodies. Then, the distractor 109 and the threaded distractor posts 101 may be removed. As shown in FIG. 7, a cervical plate, as generally indicated at 135, is affixed to the anterior of the vertebrae bodies VB above and below disc space by means of bone screws 137 so as to stabilize the operative level and enhance the fusion rate.

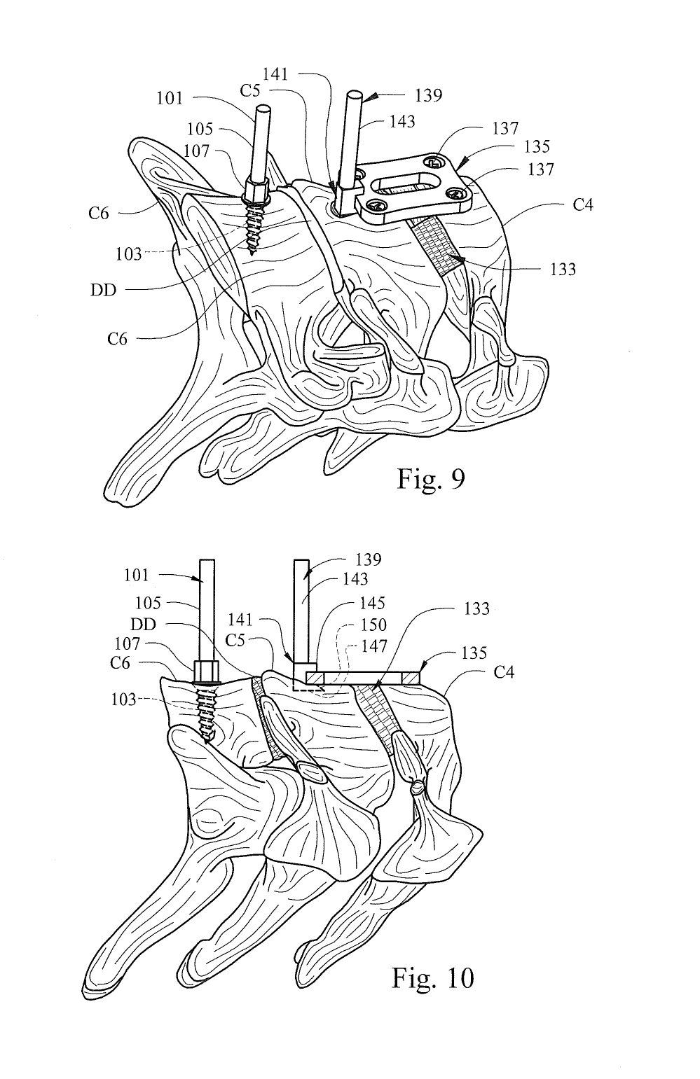

Referring now to FIGS. 9-13, the distraction system and method of the present disclosure is illustrated for reoperative procedures of a cervical segment adjacent to a segment that has previously undergone spinal fusion surgery, such as described above. As shown in FIG. 9, for example, the C4/C5 disc space has previously undergone fusion surgery with this disc space having an interbody implant 133 inserted into the disc space between C4 and C5. A conventional cervical plate 135 is fixed to C4 and C5 by screws 137. The diseased disc DD is between the adjacent segment of C5/C6. As further shown in FIG. 9, in accordance with the present disclosure, a conventional distractor post 101 is imbedded into the vertebrae body of C6 along the midline in the conventional manner. However, because of the cervical plate 135 spanning the previously surgically repaired disc space between C4 and C5, the cervical plate 135 would interfere with the installation of a conventional distractor post 101 in C5. In many instances, the installation of such a conventional distractor post in C5 would have necessitated the removal of the plate 135.

In accordance with the system and method of the present disclosure, a modified distractor member, as indicated in its entirety at 139, is adapted to be removably attached to the proximate end of cervical plate 135. Distractor member 139 has an attachment body or clevis 141 on its lower end and a post 143 extending from the attachment body. Attachment body 141 comprises an upper and a lower blade or tang, as indicated at 145, 147, respectively, which are spaced apart a distance sufficient to snugly, yet readily receive the adjacent edge of plate 135. As shown in FIG. 10, the lower blade 147 has a chisel end, as indicated at 149, so that it may be wedgingly inserted under the proximate edge of plate 135 so that the inner faces of blades 145 and 147 bear on the outer and inner faces of the with sufficient clearance to allow the attachment body to be slid onto the plate, with the throat of the attachment body engaging the edge of the plate. In this manner, distractor post 139 is securely, but removably secured to the proximate edge of the previously installed cervical plate 135 such that the post 143 may be received in a respective tube 131 of distractor instrument 109 so that a distraction force may be applied to vertebrae body C5 via post 143. It will be appreciated that upon operation of distractor instrument 109 so as distract the diseased disc space, the attachment or clevis 141 pushes against the proximate edge of the cervical plate. It will be further appreciated that if the distractor 109 applies a couple to post 143, the spacing between the inner faces of blades 145, 147 and the length of these blades is such that the blades will effectively resist such couple, and yet permit the distractor post 139 to capture the cervical plate 135 and enable the ready removal of the distractor post 139 from the cervical plate.

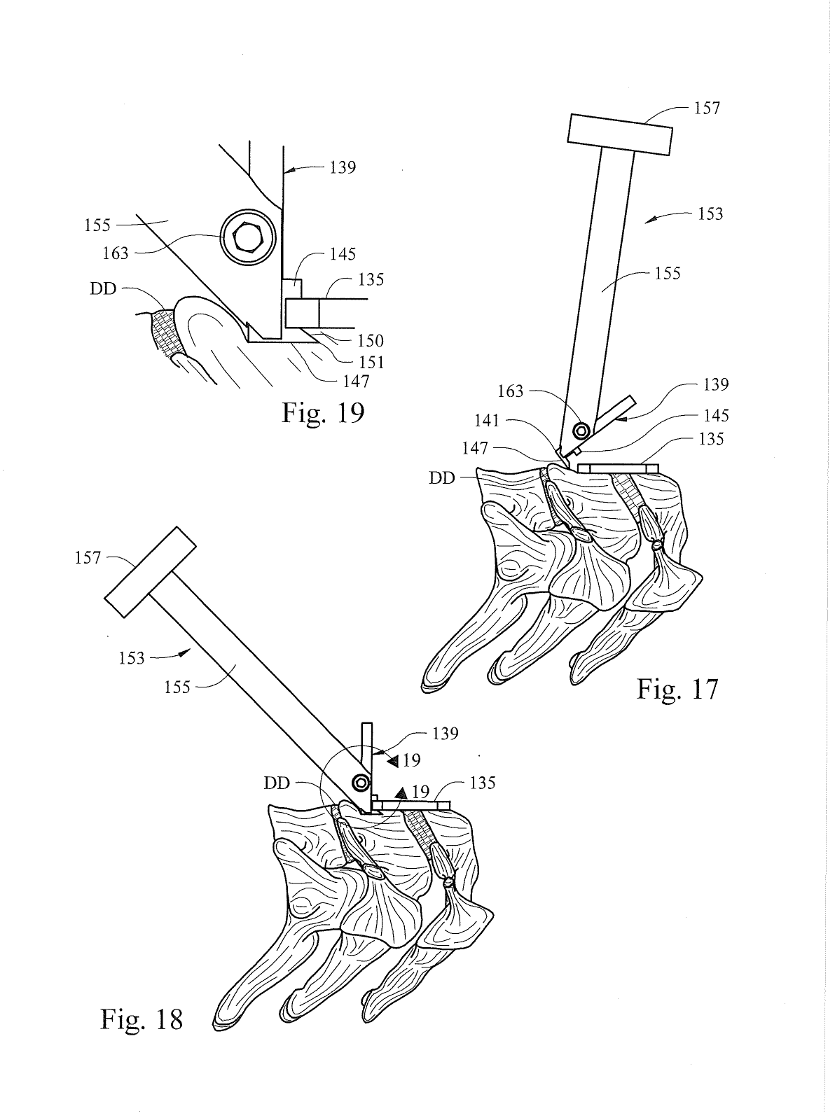

As shown in FIGS. 10, 14, 17-19, bottom blade 147 has a chisel face 150 with a sharp chisel edge 151, which upon insertion of bottom blade 147 under the proximate edge of plate 135 cuts a track or groove in the vertebrae body VB under the existing cervical plate 135 and helps guide the bottom blade underneath plate. It will be particularly noted that the bottom blade is sufficiently thin so that it does not cause the plate screws 137 to pull out of the vertebrae bodies as the post 139 is installed on the plate. Further, by cutting a track in the vertebrae body as the bottom blade is inserted under the plate, that is as the bottom blade is “toed in” to the vertebrae, the outward force that the bottom blade may apply to plate 135 as it is inserted under the plate minimizes the tendency of the bottom blade that may cause the screws 137 to pull out of the vertebrae body. Thus, the two blade design of the applicator body 141 securely and rigidly captures the adjacent edge of plate 135. It will also be noted that the upper blade 141 is somewhat shorter than the bottom blade 147 (for example, about 2 mm. shorter) to better facilitate the lower blade to be toed in under the edge of plate 135 so as to cut the above-mentioned track or groove in the vertebrae. This allows the surgeon to tap the lower blade into place, while the post 139 is angled (as shown in FIG. 17) without the upper blade contacting the cervical plate 135. Still further, it will be appreciated that the depth of the throat 148 prevents overly aggressive purchase of the chisel edge 151 during the toe-in maneuver (as shown in FIG. 17) and the depth of the throat limits how far the attachment member 141 can be driven under the plate, which, in turn, lessens the tendency of the screws 137 from being pulled out of the bone. It will be further appreciated that with distractor member 139 removably installed on the adjacent edge of plate 135, as above described, distraction of the adjacent disc space is accomplished by applying distraction force to the plate, which distributes the distraction to the four screws 137.

Even if the proximate edge of the previously applied cervical plate 135 is flush with new symptomatic disc space, a distractor member 139 of the present disclosure could still be applied to the plate, as above described, and its low profile would not obscure surgeon's vision or operative corridor while performing discectomy and decompression on the diseased disc DD undergoing surgery (i.e., on the disc of interest). It will also be appreciated that the post 143 of distraction member 139 is located generally on the midline of the vertebrae body and is substantially in line with conventional distraction post 101 installed on the vertebrae body on the opposite side of the diseased disc (e.g., installed on C6, as shown in FIG. 9), where it is substantially perpendicular to spine thus providing the most effective distraction to disc space while avoiding soft tissue banks on either side of wound.

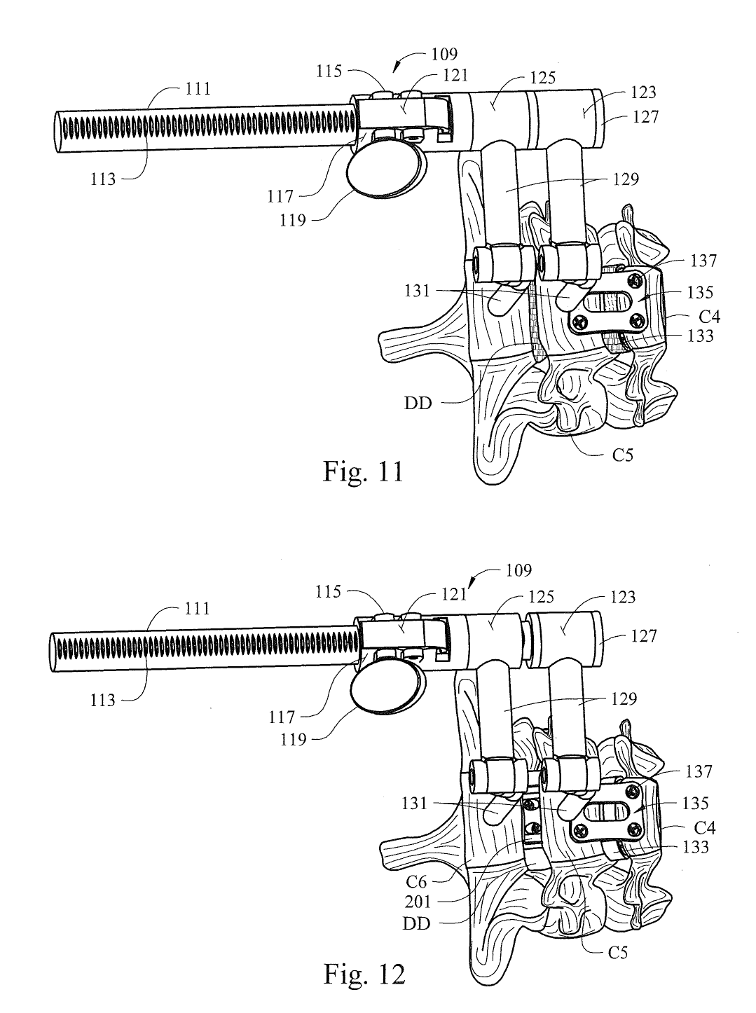

As shown in FIGS. 11 and 12, distractor instrument 109 having post receiving tubes 131 is applied to the post 143 of distractor member 139 and to post 105 of a conventional distractor post 101, applied a distractive force to the adjacent symptomatic disc space between C5/C6 facilitating discectomy of the diseased disc space DD, decompression of the spinal cord and nerve roots, preparation of fusion bed, and insertion of a zero-profile plate cervical interbody implant, as generally indicated at 201.

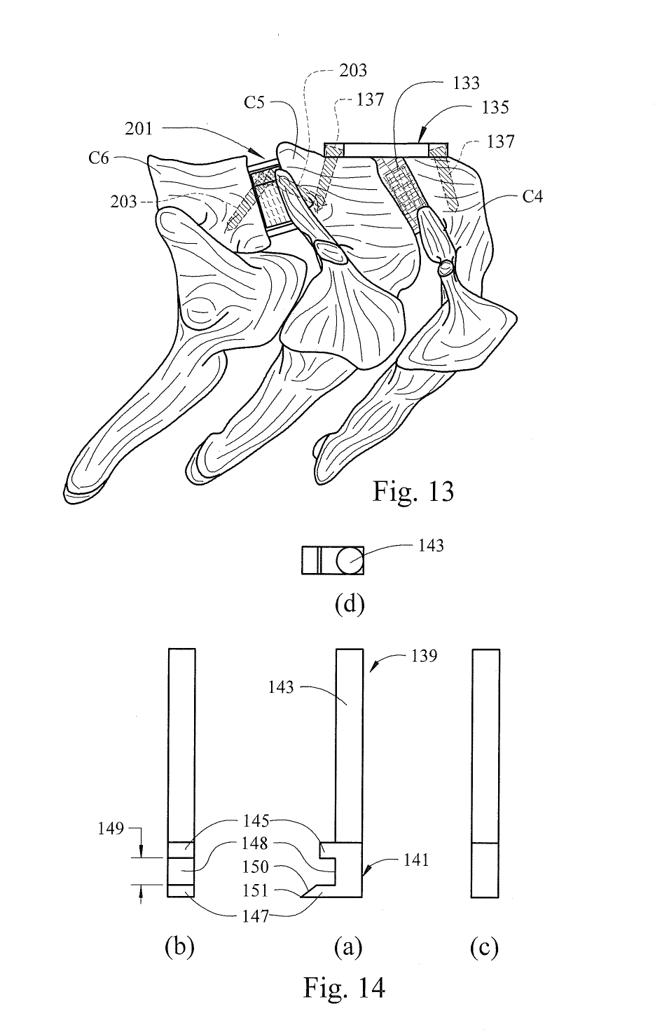

Turning now to FIG. 13, a section of the cervical spine C including, for example, vertebrae bodies C4-C6, is illustrated. The previously operated upon disc D between C4/C5 is shown to have an interbody implant 133 installed there in and a cervical plate 135 applied across the C4/C5 disc and attached (screwed) to the anterior C4/C5 vertebrae bodies. FIG. 13 further illustrates a conventional zero-profile plate cervical interbody implant 201 installed in the disc space DD between C5/C6. This zero-profile interbody implant 201 is held in place by means of bone screws 203 (as shown in dotted lines in FIG. 13) driven at an angle into vertebrae bodies C5 and C6 to hold the implant in place. It will be particularly noted that with such zero-profile implants, no external cervical plate need be installed spanning the C5/C6 disc space. Such zero-profile implants are commercially available and one such implant, known as the AVS® Anchor-C Cervical Cage System is available from Stryker Spine, Kalamazoo, Mich.

Upon completion of the installation of the zero-profile implant 201 in the disc space, the distractor instrument is removed from the post 104 of the conventional distractor post 101 and from the post 143 of distractor member 139 of the present disclosure. The distractor post 101 is removed from the vertebrae body (e.g., C6), and the distractor member 139 is removed from plate 135, as by sliding the attachment body 141 from the edge of the plate. Then, the surgical wound is closed in a standard multi-layered anatomic fashion.

Referring now to FIGS. 14a-14d, distraction member 139 is shown in greater detail. As indicated at 149 in FIG. 14b, the space or gap (also referred as a throat) between the inner faces of blades 145 and 147 is preferably only somewhat (e.g., a few hundredths of a millimeter) greater than the thickness of cervical plate 135 so that the edge of the plate may be readily received within the gap and so that when the edge of the plate is readily captured between the inner faces of the blades 145 and 147 of attachment body 141 so that the plate readily fits within this space. However, the inner faces of the blades are sufficiently snug with the outer and inner faces of the plate that rocking of the post 139 relative to the plate is minimized. Lower blade 147 has a chisel edge 151 that wedges under the cervical plate and acts to cut a track (a groove) or depression in the vertebrae body as the post 139 is installed, thereby to minimize the pull out force exerted on screws 137 holding the plate 135 as the lower blade is driven between the vertebrae body and the inner surface of plate 135. It will also be noted in FIGS. 14a and 14b that the back face 148 of throat 149 engages the edge of plate 135 and thus limits how far the distraction member 139 may be driven onto the edge of plate 135, again minimizing the tendency of the plate to pull out of the vertebrae to which it is attached.

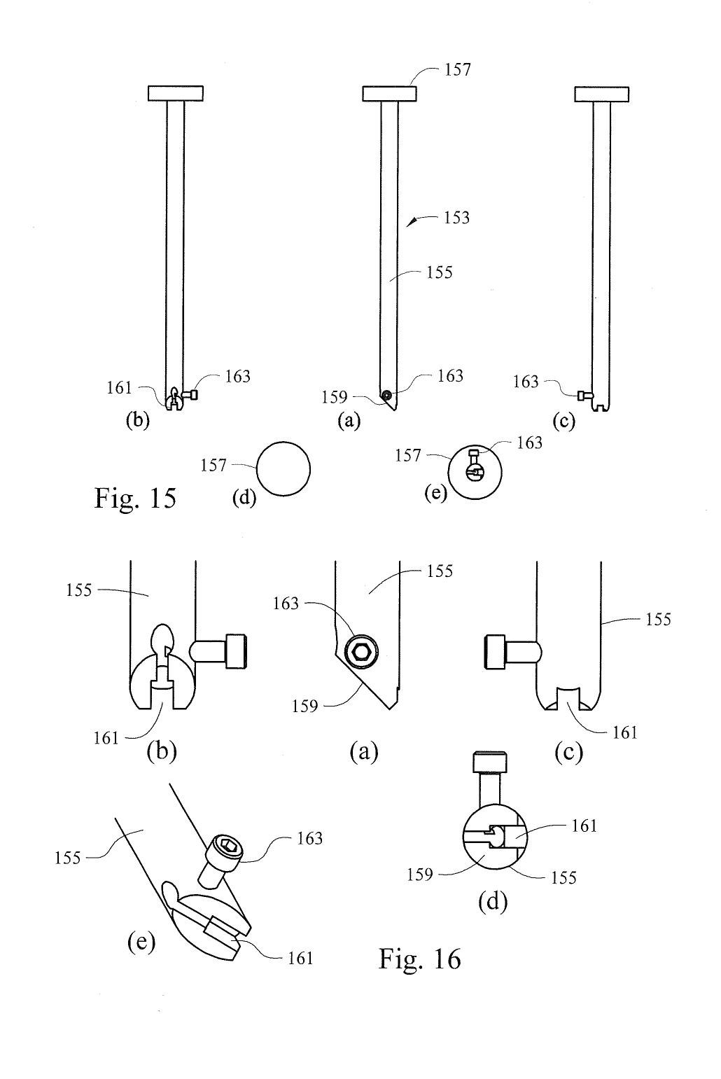

Referring now to FIGS. 15-19, an applicator or instrument for applying distractor member 139 onto a previously installed plate 135 is shown in its entirety at 153. As shown in FIGS. 15a-15e, applicator 153 has an elongate cylindrical body 155 having a pommel handle 157 on its outer end for purposes as will appear. The distal end of body 155 is provided with a beveled end face 159 angled at about 45° to the axis of the cylindrical body. This beveled end face has a slot 161 (as best shown in FIGS. 16a-16e) therein sized and shaped to receive the attachment body 141 and post 143 of distraction member 139. A set screw 163 is carried on the distal end of the cylindrical body 155 such that upon installation of a distractor member 139 in slot 161 and such that upon the set screw being tightened against the distractor member, the distractor member is firmly held within the slot, as perhaps best shown in FIGS. 17-19.

The distractor member 139 is installed on plate 135 in the manner illustrated in FIGS. 17-19. As shown in FIG. 17, with a distractor member 139 inserted in slot 161 and firmly held in place by set screw 163, the surgeon may install the distractor member onto and adjacent edge of a previously installed cervical plate 135 by positioning the attachment member 141 of the distractor member relative to the edge of the plate in the manner shown in FIG. 17. The surgeon then maneuvers the attachment body 141, such as by rotating the applicator 153 from the position shown in FIG. 17 to the position generally shown in FIG. 18 such that chisel edge 151 of the lower blade 147 is in position to glide along the ventral spine toward the edge of the plate such that the chisel edge is in position to be inserted (wedged) between the underside of the plate and the vertebrae body with the thickness of the plate is generally in register with gap 149. Then, the surgeon may tap the pommel handle 157 with a hammer so that the chisel blade is gently driven between the vertebrae body and the underside of plate 135. The chisel edge 151 will cut a track or a groove in the vertebrae body as the chisel edge is driven under the plate so that the pull off or pry out force exerted by the chisel blade on the plate is minimized. With the distractor member 139 so installed on the plate 135 in the manner shown in FIG. 18, the set screw 163 is loosened and the application tool is removed from the distractor member 139, leaving the distractor member installed on the cervical plate. With the edge of the plate captured within gap 149 of the attachment body 141, the post 143 is rigidly secured to the plate by the inner faces of the blades 145, 147. With the distractor member 139 so installed on the edge of plate 135, with the post 143 received in a respective distraction tube 131 of distractor instrument 109, and with the post 105 of the threaded distractor post 101, the distractor instrument may be operated so as to distract the disc space such that the distraction force is transmitted to the vertebrae body (e.g., C5) by means of the cervical plate 135 and the screws 137 holding the plate onto the vertebrae body. Of course, at the appropriate time, the distraction member 139 may be readily removed from the plate 135 merely by pulling or sliding the attachment body 141 free of the plate.

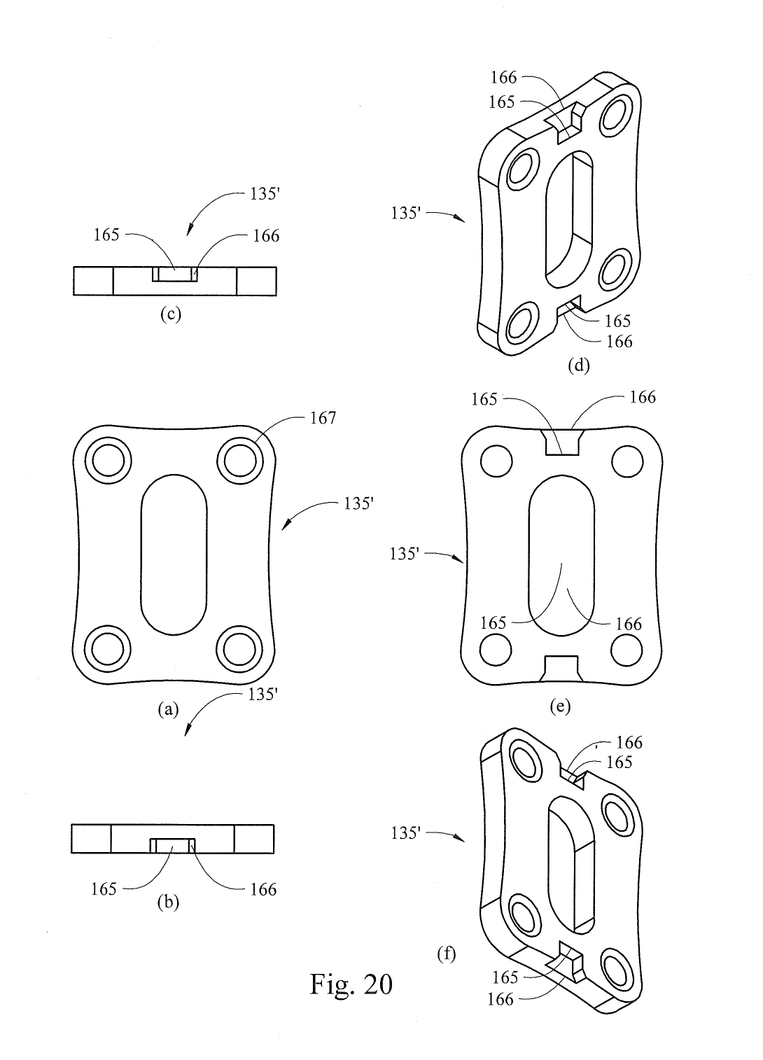

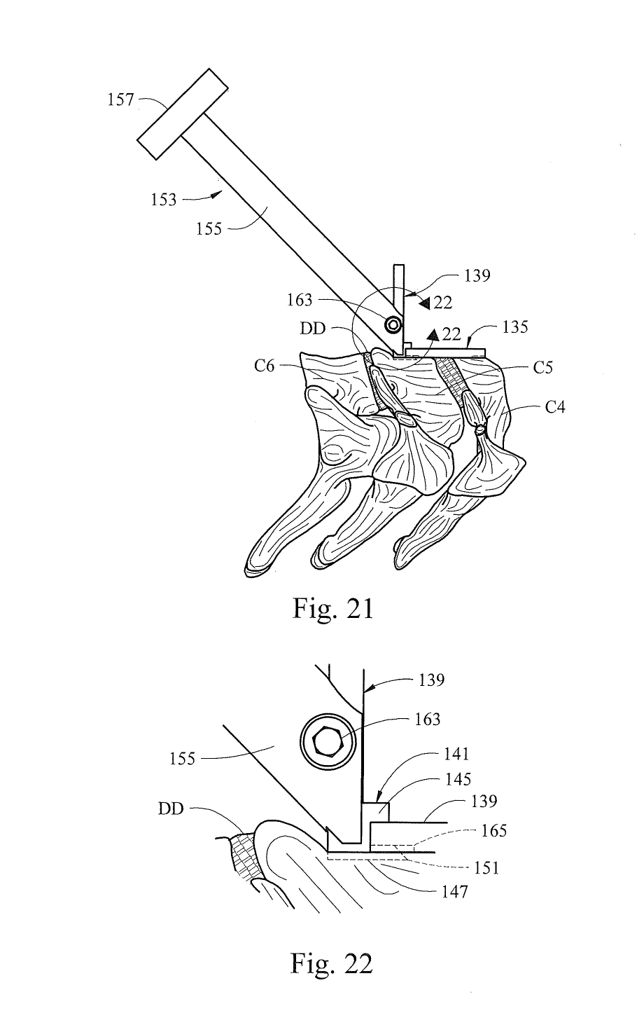

Referring now to FIG. 20, a cervical plate modified in accordance with the instant disclosure is indicated in its entirety at 135′. Specifically, it will be noted that on the underside of plate 135′ at the superior and inferior ends of the plate, a slot 165 is provided. This slot 165 cooperates with lower blade 147 of attachment body 141 of post 139 so that the slot readily receives at least a portion of the height of the lower blade. This, in turn, reduced the force applied to the plate that would tend to pull the plate screws 137 from its respective vertebrae body. This will facilitate the installation of the distractor member 139 on to the plate 135′. In addition, plate 135′ has four screw holes 167 to facilitate mounting of the plate on the anterior respective vertebrae bodies. It will be understood that the gap 149 of distractor member 139 would be adjusted to reflect the change in the thickness of the plate 135′ at the level of the slot 165. As shown at 166, the open end of slot 165 may be wider than the rear of the slot so as to guide the entrance of the blade 147 into the slot.

It will be further understood that in accordance with the present disclosure, other modifications to the plate 135 are envisioned that would allow removable attachment of a distractor post to the plate. For example, the portion of the plate proximate the superior and inferior ends of the plate may be provided with a threaded hole (not shown). This threaded hole may have a threaded plug installed therein. Then, in the event that reoperative surgery is required several years later on an adjacent spine segment, for installation of a distraction post on the cervical plate, the threaded plug may be removed and a threaded distraction post may be installed on the plate to enable distraction of the adjacent segment without having to remove the plate. Once the reoperative surgery is nearly complete, the distractor post inserted into the threaded hole is removed and a threaded plug may be re-installed. The purpose of the threaded plug is to insure that the threads in the hole are free of debris that would interfere with the installation of the threaded distractor post in the holes. This enhancement would be present on a particular cervical plate product line embodying the modified plate 135′ and would facilitate the use of a zero-profile plate cervical implant in the future event of adjacent segment disc disease.

The surgical technique of the present disclosure involves forming an anterior cervical incision using standard anatomic surface landmarks. This incision is usually transverse and centered over desired disc space level. For a right-handed surgeon, this will be to the right side of the patient's neck. After making the incision, meticulous sharp and blunt dissection using an interfascial technique dividing the superficial, middle, and deep cervical fascia sequentially staying medial to carotid artery and lateral to the trachea and esophagus. Because with reoperation procedures, scar tissue will obscure many of the natural dissection planes, the interfascial corridor that normally easily separates with blunt finger dissection will likely not exist. The surgeon must very carefully dissect through the scar tissue identifying normal anatomic structures and paying great attention not to injure critical structures, such as the carotid artery, trachea and esophagus. Fluoroscopic localization, digital palpation, and direct visual confirmation of previously applied edge of the anterior cervical plate 135 will help localize the desired adjacent symptomatic disc space: A side-to-side, self-retaining retractor (not shown) is applied with its blades anchored beneath the longus colli muscles. Then, an applicator 153 having a distractor member 139 installed in slot 161 is then toed into place (as shown in FIGS. 17 and 18) by gently striking pommel handle 157 and driving chisel edge 151 of the bottom blade 147 beneath the adjacent portion of plate 135. The post is then brought perpendicular to spine (as shown in FIG. 18) and driven forward again by striking the pommel handle so that the two blades 145 and 147 capture the edge of the plate 135. The set screw 163 is then operated to unlock post 139 from the applicator. A standard threaded distractor post 101 is then impacted and screwed into place on the other side of the disc space, such as in C6, as shown in FIG. 10. A cervical distractor instrument 109 is then applied to the posts 105 and 143 and the distractor is operated so as to distract the disc space.

Standard cervical discectomy of the distracted disc space is then performed, followed by endplate preparation for fusion, decompression of spinal cord and nerve roots, depth gauge to measure for implant and screw length, and finally trialing for the implant 201.

The central hollow core of the zero-profile plate cervical implant 201 is packed with bone graft, and is then impacted into place flush with the anterior margin of the spine, as shown in FIG. 13. Screws 203 are then used to fixate the implant to the spine, as shown in FIG. 13. A standard post applicator is then used to remove/unscrew standard post 101 from, for example, vertebrae C6, as shown in FIG. 9, and the distractor post 139 of the present disclosure is slid from the end of cervical plate 135 using a standard post applicator. Standard anatomic closure is then performed.

As various changes could be made in the above constructions and methods without departing from the scope of the disclosure, it is intended that all matter contained in the above description or shown in the accompanying drawings shall be interpreted as illustrative and not in a limiting sense.

1. A system for distracting a disc space in a cervical spine at a level adjacent to a previously operated disc space where, in the previous surgery, a cervical plate has been applied to the anterior spine to span the previously operated upon disc space and where the cervical plate is attached to the vertebrae bodies on opposite sides of the previously operated upon disc space where the cervical plate covers at least a portion of the vertebrae bodies, a threaded distractor post threadably secured to a third vertebrae body delimiting said disc space to be operated upon, and a distractor member fixedly and removably secured to said cervical plate, said distractor post and said distractor member having a post extending generally perpendicularly to the midline of the cervical spine and projecting anteriorly of the cervical spine so that a conventional distractor may be applied to the posts of both the distractor post and the distractor member for applying a distraction force to the vertebrae bodies delimiting the disc space to be operated upon.

2. The system of claim 1 wherein said distractor member has an attachment body on its distal end, said attachment body having a lower and an upper blade spaced from one another so as to capture a portion of said plate therebetween as said attachment body is slid onto a proximate edge of said cervical plate so that a distraction force may be applied to the post of said distractor member thereby enabling distraction of the disc space to be operated upon without removal of said plate.

3. The system of claim 2 wherein said lower blade has a beveled chisel edge for being wedgingly insertable between said vertebrae body and the underside of said cervical plate.

4. The system of claim 2 further comprising an applicator for applying said distractor member to said cervical plate, said applicator having an elongate body, the distal end of which has a slot for receiving one of said second distractor posts with said attachment body held relative to said elongate body so that the proximate edge of said cervical plate may be received between said upper and lower blades of said attachment body.

5. The system of claim 4 wherein with said applicator positioned such said blades of said attachment body of said distractor member are disposed to receive said edge of said cervical plate, said applicator may be operated by the surgeon to force said lower blade between the underside of said plate and the vertebrae body so that the edge of the cervical plate is captured between said blades.

6. The system of claim 5 wherein said lower blade has a chisel edge which at least in part cuts a track in said vertebrae body as said lower blade is forced between said plate and said vertebrae body.

7. The system of claim 4 wherein said cervical plate has a slot in its inner face for at least in part receiving said lower blade of said attachment body as said distractor member is installed on said cervical plate.

8. The system of claim 7 wherein the proximate end of said slot is wider than the distal end so as to guide the lower blade of the attachment body into the slot.

9. A system for distracting a disc space in a cervical spine at a level adjacent to a previously operated disc space where, in the previous surgery, a cervical plate has been applied to the anterior of the spine to span the previously operated upon disc space and where the cervical plate is attached to the vertebrae bodies on opposite sides of the previously operated upon disc space such that the cervical plate covers at least a portion of the vertebrae body delimiting the disc space to be operated upon, a threaded distractor member secured to another vertebrae body delimiting said disc space to be operated upon, and a second distractor member fixedly and removably attached to said cervical plate, both of said distractor members having a post extending generally perpendicularly to the midline of the cervical spine and projecting anteriorly of the cervical spine so that a conventional distractor may be applied to the posts of both distractor members for applying a distraction force to the vertebrae bodies delimiting the disc space to be operated upon.

10. The system of claim 9 wherein said second distractor member has an attachment body on its distal end, said attachment body having a lower and an upper blade spaced from one another so as to capture a portion of said plate therebetween as said attachment body is slid onto a proximate edge of said cervical plate so that a distraction force may be applied to the posts of both of said distractor members thereby enabling distraction of the disc space to be operated upon without removal of said plate.

11. The system of claim 10 further comprising an applicator for applying said second distractor member to said cervical plate, said applicator having an elongate body, the distal end of which is configured to receive one of said second distractor posts with said attachment body held relative to said elongate body so that the proximate edge of said cervical plate may be received between said upper and lower blades of said attachment body.

12. The system of claim 11 wherein with said applicator positioned such said blades of said attachment body of said second distractor member are disposed to receive said edge of said cervical plate, said applicator may be operated by the surgeon to force said lower blade between the underside of said plate and the vertebrae body so that the edge of the cervical plate is captured between said blades.

13. A method of distracting a disc space in the cervical spine at an adjacent symptomatic level to a disc space that has been previously operated upon where in such previous operation a cervical plate has been secured to the anterior of the cervical vertebrae bodies on opposite sides of the previously operated upon disc space, said cervical plate covering at least a portion of the anterior vertebral body of the vertebrae that delimits the disc space to be operated upon, said method comprising the steps of:
installing a threaded distraction post to another vertebrae body delimiting said disc space to be operated upon;
removably attaching a distractor member to said cervical plate, where both said distractor post and said distractor member have a post extending anteriorly from approximately the midline of said cervical spine;
applying a distractor instrument to said posts; and
operating said operating distractor instrument so as to distract the disc space to be operated upon.

14. The method of claim 13 wherein the step of removably attaching the distractor member to said cervical plate comprises capturing a portion of said cervical plate between a pair of spaced blades on the lower end of said distractor member where said blades removably mount said distractor member on said cervical plate such that a distraction force may be applied to the vertebrae body delimiting said disc space to be distracted via said cervical plate.

15. The method of claim 13 wherein the step of removably attaching the distractor member to said cervical plate comprises capturing a portion of said cervical plate between a pair of spaced blades on the lower end of said distractor member where said blades bear against the outer and inner surfaces of the portion of the cervical plate captured between said blades such that a distraction force may be transmitted to the vertebrae body to delimiting said disc space via the cervical plate.

16. The method of claim 13, wherein after distraction of the disc space being operated upon, further comprising the steps of: performing discectomy and decompression of the diseased disc space; and installing a zero-profile plate interbody implant packed with bone graft material into the diseased disc space.
